(19) United States
(12) Patent Application Publication
(10) Pub. No.: US 2001/0051636 A1
(43) Pub. Date: Dec. 13, 2001

(54) Combination treatment for inhibiting bone loss

(76) Inventors: Larry J. Black, Indianapolis, IN (US); George J. Cullinan, Trafalgar, IN (US)

Correspondence Address: DAN L. WOOD, Eli Lilly And Company, Lilly Corporate Center, Patent Division/DLW, Indianapolis, IN, 46285, US

(21) Appl. No.: 09/520,737

(22) Filed: Mar. 8, 2000

Related U.S. Application Data
(63) Continuation of application No. 08/279,363, filed on Jul. 22, 1994, now abandoned.

(51) Int. Cl. A61K 31/445

(52) U.S. Cl. 514/320; 514/422; 514/443

(57) ABSTRACT

The present invention provides a method for inhibiting bone loss comprising administering to a human in need thereof a first compound selected from 1) triarylethylenes; 2) 2,3-diaryl-2H-1-benzopyrans, 3) 1-aminoalkyl-2-phenylindoles; 4) 2-phenyl-3-aroylbenzothiophenes, 5) 1-substituted-2-aryl-dihydronaphthalenes; or 6) benzofurans, and a second compound being a bisphosphonate: or pharmaceutically acceptable salts and solvates thereof. Also encompassed by the invention are combination pharmaceutical formulations and salts.

### BACKGROUND OF THE INVENTION

Current major diseases or conditions of bone which are of public concern include post-menopausal osteoporosis, senile osteoporosis, patients undergoing long-term treatment of corticosteroids, side effects from glucocorticoid or steroid treatment, patients suffering from Cushings's syndrome, gonadal dysgensis, periarticular erosions in rheumatoid arthritis, osteoarthritis, Paget's disease, osteohalisteresis, osteomalacia, hypercalcemia of malignancy, osteopenia due to bone metastases, periodontal disease, and hyperparathyroidism. All of these conditions are characterized by bone loss, resulting from an imbalance between the degradation of bone (bone resorption) and the formation of new healthy bone. This turnover of bone continues normally throughout life and is the mechanism by which bone regenerates. However, the conditions stated above will tip the balance towards bone loss such that the amount of bone resorbed is inadequately replaced with new bone, resulting in net bone loss.

One of the most common bone disorders is post-menopausal osteoporosis which affects an estimated 20 to 25 million women in the United States alone. Women after menopause experience an increase in the rate of bone turnover with resulting net loss of bone, as circulating estrogen levels decrease. The rate of bone turnover differs between bones and is highest in sites enriched with trabecular bone, such as the vertebrae and the femoral head. The potential for bone loss at these sites immediately following menopause is 4-5% per year. The resulting decrease in bone mass and enlargement of bone spaces leads to increased fracture risk, as the mechanical integrity of bone deteriorates rapidly.

At present, there are 20 million people with detectable vertebral fractures due to osteoporosis and 250,000 hip fractures per year attributable to osteoporosis in the U.S. The latter case is associated with a 12% mortality rate within the first two years and 30% of the patients will require nursing home care after the fracture. Therefore, bone disorders are characterized by a noticeable mortality rate, a considerable decrease in the survivor's quality of life, and a significant financial burden to families.

Essentially all of the conditions listed above would benefit from treatment with agents which inhibit bone resorption. Bone resorption proceeds by the activity of specialized cells called osteoclasts. Osteoclasts are unique in their ability to resorb both the hydroxyapatite mineral and organic matrix of bone. They are somewhat similar to the cartilage resorbing cells, termed chondroclasts. It is for this reason that potent inhibitors of osteoclastic bone resorption may also inhibit the cell-mediated degradation of cartilage observed in rheumatoid arthritis and osteoarthritis.

Therapeutic treatments to impede net bone loss include the use of estrogens. Estrogens have been shown clearly to arrest the bone loss observed after menopause and limit the progression of osteoporosis; but patient compliance has been poor because of estrogen side-effects. These side effects include resumption of menses, mastodynia, increase in the risk of uterine cancer, and possibly an increase in the risk of breast cancer.

Alternatively, calcitonin has been used to treat osteoporotic patients. Salmon calcitonin has been shown to directly inhibit the resorption activity of mammalian osteoclasts and is widely prescribed in Italy and Japan. However, calcitonins are prohibitively expensive to many and appear to be short-lived in efficacy. That is, osteoclasts are able to “escape” calcitonin inhibition of resorption by down-regulating calcitonin receptors. Therefore, recent clinical data suggest that chronic treatment with calcitonin may not have long term effectiveness in arresting the post-menopausal loss of bone. There continues to be great interest in research directed to novel therapies to inhibit bone loss.

### SUMMARY OF THE INVENTION

This invention provides a novel method for inhibiting bone loss comprising administering to a human in need thereof a first compound selected from 1) triarylethylenes; 2) 2,3-diaryl-2H-1-benzopyrans, 3) 1-aminoalkyl-2-phenylindoles; 4) 2-phenyl-3-aroylbenzothiophenes, 5) 1-substituted-2-aryl-dihydronaphthalenes; or 6) benzofurans; and a second compound being a bisphosphonate, or pharmaceutically acceptable salts and solvates thereof.

Also encompassed by the invention are combination pharmaceutical formulations and salts.

### DETAILED DESCRIPTION OF THE INVENTION

This invention concerns the discovery that combination therapy for humans, comprising administering a component from the first group of compounds, as defined above, with a bisphosphonate, is useful in the inhibition of bone loss. The therapy may be sequential, concurrent or simultaneous, with the latter two being preferred.

The general chemical terms used in the description of the compounds of this invention have their usual meanings. For example, the term “alkyl” by itself or as part of another substituent means a straight or branched chain alkyl radical having the stated number of carbon atoms such as methyl, ethyl, propyl, and isopropyl and higher homologues and isomers where indicated.

The term “alkoxy” means an alkyl group having the stated number of carbon atoms linked to the parent moiety by an oxygen atom, such as methoxy, ethoxy, propoxy, butoxy, pentyloxy, and hexyloxy and also includes branched chain structures such as, for example, isopropoxy and isobutoxy.

The term “substituted alkyl” includes an alkyl substituted once or more with substitutents known in the art. As this term is used in conjunction with the bisphosphonates, those references in this art would disclose such substitutents.

The term “C₁-C₇-alkanoyloxy” means a group —O—C(O)—R^{a} where R^{a} is hydrogen or C₁-C₆ alkyl and includes formyloxy, acetoxy, propanoyloxy, butanoyloxy, pentanoyloxy, hexanoyloxy, and the like and also includes branched chain isomers such as, for example, 2,2-dimethylpropanoyloxy, and 3,3-dimethylbutanoyloxy.

Analogously, the term C₄-C₇ cycloalkanoyloxy” means a group —O—C(O)—(C₃-C₆ cycloalkyl) where the C₃-C₆ alkyl group includes cyclopropyl, cyclobutyl, cyclopentyl and cyclohexyl.

The term “(C₁-C₆-alkoxy)-C₁-C₇-alkanoyloxy” means a group —O—C(O)—R^{b}—O—(C₁-C₆ alkyl) where R^{b} is a bond (C₁-C₆ alkoxycarbonyloxy) or C₁-C₆ alkanediyl and includes, for example, methoxycarbonyloxy, ethoxycarbonyloxy, propoxycarbonyloxy, butoxycarbonyloxy, methoxyacetoxy, methoxypropanoyloxy, methoxybutanoyloxy, methoxy-pentanoyloxy, methoxyhexanoyloxy, ethoxyacetoxy, ethoxypropanoyloxy, ethoxybutanoyloxy, ethoxypentanoyloxy, ethoxyhexanoyloxy, propoxyacetoxy, propoxypropanoyloxy, propoxybutanoyloxy, and the like.

The term “unsubstituted or substituted aroyloxy” means a group —O—C(O)-aryl where aryl is a phenyl, naphthyl, thienyl or furyl group that is, as to each group, unsubstituted or monosubstituted with a hydroxyl, halo, C₁-C₃ alkyl, or C₁-C₃ alkoxy.

The term “unsubstituted or substituted aryloxycarbonyloxy” means a group —O—C(O)—O-aryl where aryl is a phenyl, naphthyl, thienyl or furyl group that is, as to each group, unsubstituted or monosubstituted with a hydroxyl, halo, C₁-C₃ alkyl or C₁-C₃ alkoxy.

The term “halo” means chloro, fluoro, bromo or iodo.

The term “inhibit” is defined to include its generally accepted meaning which includes preventing, prohibiting, restraining, and slowing, stopping or reversing progression, or severity, and holding in check and/or treating existing characteristics. The present method includes both medical therapeutic and/or prophylactic treatment, as appropriate.

The term “pharmaceutically acceptable salts” refers to salts of the compounds of the above classes which are substantially non-toxic to living organisms. Typical pharmaceutically acceptable salts include those salts prepared by reaction of a compound of the above class with a pharmaceutically acceptable mineral or organic acid, or a pharmaceutically acceptable alkali metal or organic base, depending on the types of substituents present on the compound.

Examples of pharmaceutically acceptable mineral acids which may be used to prepare pharmaceutically acceptable salts include hydrochloric acid, phosphoric acid, sulfuric acid, hydrobromic acid, hydroiodic acid, phosphorous acid and the like. Examples of pharmaceutically acceptable organic acids which may be used to prepare pharmaceutically acceptable salts include aliphatic mono and dicarboxylic acids, oxalic acid, carbonic acid, citric acid, succinic acid, phenyl-substituted alkanoic acids, aliphatic and aromatic sulfonic acids and the like. Such pharmaceutically acceptable salts prepared from mineral or organic acids thus include hydrochloride, hydrobromide, nitrate, sulfate, pyrosulfate, bisulfate, sulfite, bisulfite, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Many compounds of the above classes which contain a carboxy, carbonyl, or hydroxy or sulfoxide group may be converted to a pharmaceutically acceptable salt by reaction with a pharmaceutically acceptable alkali metal or organic base. Examples of pharmaceutically acceptable organic bases which may be used to prepare pharmaceutically acceptable salts include ammonia, amines such as triethanolamine, triethylamine, ethylamine, and the like. Examples of pharmaceutically acceptable alkali metal bases included compounds of the general formula MOZ, where M represents an alkali metal atom, e.g. sodium, potassium, or lithium, and Z represents hydrogen or C₁-C₄ alkyl.

It should be recognized that the particular anion or cation forming a part of any salt of this invention is not critical, so long as the salt, as a whole, is pharmacologically acceptable and as long as the anion or cationic moiety does not contribute undesired qualities.

In addition, some of the compounds disclosed as useful in the methods of the present invention may form solvates with water or common organic solvents. Such solvates are included within the scope of the present invention and solvates thereof.

The class of compounds known as bisphosphonates includes those compounds which contains a di-phosphonic acid moiety separated by a carbon link and include a variety of side-chains, usually containing a basic function. The compounds have the following general structure:

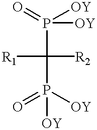

Y₁, R₁ and R₂ may be those substitutents as defined in U.S. Pat. No. 5,139,786, and EPO Publication 0416689A2, published Mar. 13, 1991, incorporated herein by reference, although not limited to such.

Pharmacologically, these compounds have been shown to slow or stop bone resorption by inhibiting osteoclast cell function. Several compounds of this class are currently undergoing clinical evaluation for the treatment of post-menopausal osteoporosis. Many of these compounds are also being evaluated for the treatment of Paget's Disease and hypercalcemia malignancy and several have been approved.

The art refers to three different generations of bisphosphonates. The first generation usually refers to the compound etidronate. This compound is being marketed for the treatment of Paget's disease and hypercalcemia malignacy.

The second generation of bisphosphonates refers to the compounds clodronate and pamidronate. Clodronate is marketed for Paget's disease and hypercalcemia maligancy. Pamidronate will probably be approved for osteoporosis in some European countries in the near future.

The third generation of bis-phosphonates refer to alendronate, residronate, and tiludronate and a host of lesser known compounds. Pharmacologically, these compounds are much more potent and are claimed to have fewer side-effects.

The structures of some bisphosphonate compounds are as follows:

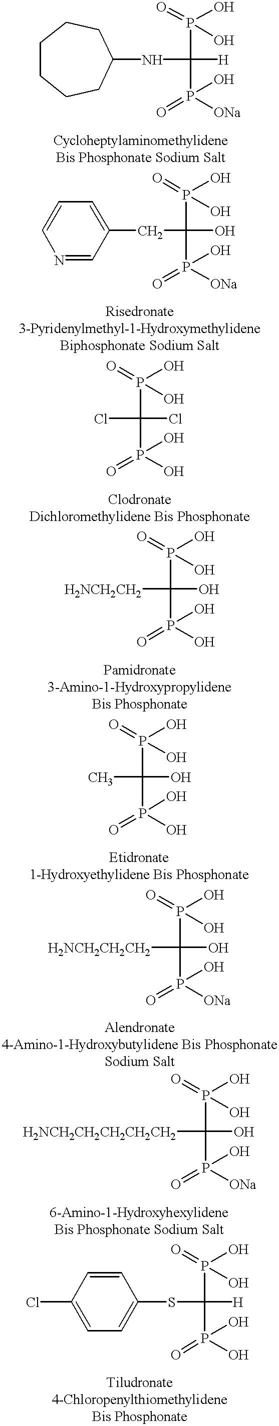

Preferred are alendronate, pamidronate, risedronate, cycloheptylaminomethylidene bis phosphonate, and 3-pyrolidenyl-1-hydroxy-propylidene bisphosphosphonate, and salts and solvates thereof.

Bisphosphonates appear to have the potential of treating osteoporosis; however, they also appear to have potential detrimental side-effects:

1) they have the potential of inhibiting bone formation as well as resorption;

2) they are poorly adsorbed via oral adminstration and are known to cause G.I. irritation;

3) they have extremely long half-lives in bone;

4) they may all have the potential for causing osteomalacia; and

5) there is concern as to the bio-mechanical strength of the bones treated with bis-phosphonates.

In general, it is felt that these compounds may have great promise for treating osteoporosis; however, there is concern as to their long term effects

Therefore, it is reasonable that the minimal exposure of the osteoporotic patient to these compounds would be desirable. Reducing exposure without sacrificing efficacy might be achievable by either using the bis-phosphonates for a limited period of time (cyclically) or by enhancing their efficacy by the inclusion of another anti-resorptive agent, working by a different mechanism of action.

The first class of compounds to be therapeutically combined with a bisphosphonate comprises triarylethylenes. These compounds are widely known and are disclosed in and prepared according to procedures described in U.S. Pat. No. 4,536,516; U.S. Pat. No. 2,914,563; Ogawa, et al., Chem. Pharm. Bull., 39(4), 911 (1991) which are all incorporated by reference herein, in their entirety. Specific illustrative compounds within this class include Tamoxifene, Clomiphene and (Z)-4-[1-[4-[2-dimethylamino)ethoxy]phenyl]-2-(4-isopropylphenyl)-1-butenyl]phenyl monophosphate.

The triarylethylenes include compounds having the formula

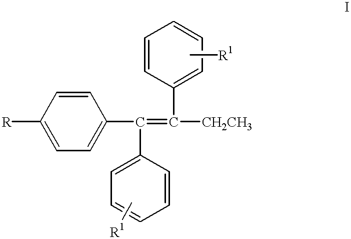

where R is a basic ether group of the formula —OCₙH₂ₙA where n is 2, 3 or 4 and A is a dialkylamino group where the alkyl groups independently contain from 1 to 4 carbon atoms or a cyclic structure selected from N-piperidinyl, N-pyrrolidinyl, N-morpholinyl, and N-hexamethyleneimino; each R¹ is independently hydrogen, hydroxy, halogen or methoxy; and pharmaceutically acceptable salts and solvates thereof.

U.S. Pat. No. 4,536,516 describes Tamoxifene, a triarylethylene having the formula

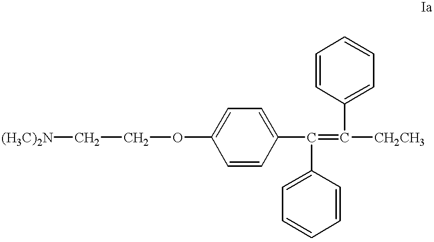

and pharmaceutically acceptable acid addition salts and solvates thereof, and discloses methods of synthesis.

Similarly, U.S. Pat. No. 2,914,563 describes triarylethylenes having the formula

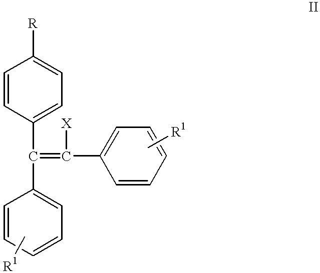

where

R is a basic ether group of the formula —OCₙH₂ₙA where n is 2,3 or 4 and A is a dialkylamino group where the alkyl groups independently contain from 1 to 4 carbon atoms or a cyclic structure such as N-piperidinyl, N-pyrrolidinyl or N-morpholinyl group. The group —OCₙH₂ₙA is bonded to the phenyl ring para to the carbon atom bonded to the ethylene group. Each R¹ is independently hydrogen, hydroxy, halogen or methoxy; X is halogen; and pharmaceutically acceptable salts and solvates thereof. Methods of synthesizing these compound are disclosed therein.

In Ogawa et al., supra, triarylethylenes are disclosed having the formula

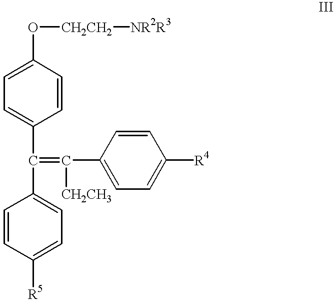

where

R² and R³ are independently selected from hydrogen and methyl;

R⁴ is isopropyl, isopropen-2-yl, or mono or dihydroxy isopropyl;

R⁵ is hydroxy or phosphate (—OPO₃H₂); and pharmaceutically acceptable salts and solvates thereof. This article also discloses synthesis of these compounds.

In addition, U.S. Pat. No. 5,254,594 and EPO 054,168 describe droloxifene, a triarylethylene having the formula

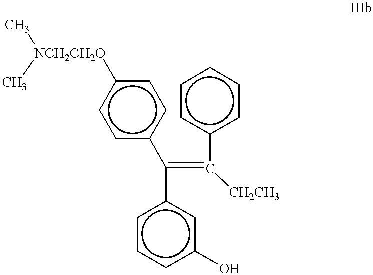

A second class of compounds comprises the 2,3-diaryl-2H-1-benzopyrans. These compounds are disclosed in and prepared according to procedures described in EP 470 310A1, and Sharma, et al., J. Med. Chem., 33. 3210, 3216, 3222 (1990) which are incorporated by reference herein in their entirety. Specific illustrative compounds within this class include 2-[4-[2-(1-piperidinyl)ethoxy]phenyl]-3-[4-hydroxyphenyl]-2H-1-benzopyran; 2-[4-[2-(1-piperidinyl)ethoxy]phenyl]-3-phenyl-7-methoxy-2H-1-benzopyran; 2-[4-[2-(1-piperidinyl)ethoxy]phenyl]-3-[4-hydroxyphenyl]-7-hydroxy-2H-1-benzopyran.

EP 470 310 A1 describes benzopyrans having the formula

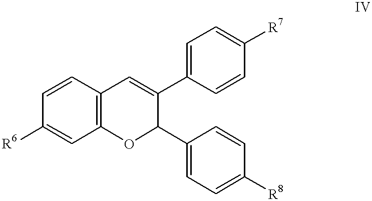

where

R⁶ and R⁷ are the same or different hydrogen hydroxy, C₁-C₁₇ alkoxy or C₂-C₁₈ alkanoyloxy;

R⁸ is

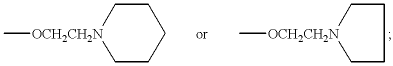

and pharmaceutically acceptable salts and solvates thereof.

Synthesis of these benzopyrans is described therein.

A third class of compounds comprises the 1-aminoalkyl-2-phenylindoles. These compounds are disclosed in and prepared according to procedures described in von Angerer, et al., J. Med. Chem., 33, 2635 (1990) which is incorporated by reference herein in its entirety.

The 1-aminoalkyl-2-phenylindoles described in von Angerer et al., supra, are those having the formula

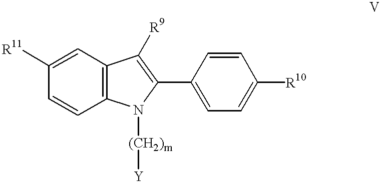

where

R⁹ is hydrogen or methyl;

R¹⁰ and R¹¹ are methoxy or hydroxy;

m is 4 to 8;

Y is NR¹²R¹³ where R¹² and R¹³ are independently selected from hydrogen, methyl and ethyl or one of R¹² or R¹³ is hydrogen and the other is benzyl or are combined with the nitrogen atom to constitute a pyrrolidinyl, piperidinyl or morpholinyl group, and pharmaceutically acceptable salts and solvates thereof. Procedures for synthesizing these compound are specifically disclosed or referenced therein.

A fourth class of compounds comprises the 2-phenyl-3-aroylbenzo[b]thiophenes; (z-triarylpropenones). These compounds are disclosed in and prepared according to procedures described in U.S. Pat. No. 4,133,814; U.S. Pat. No. 4,418,068; and Jones, et al., J. Med. Chem., 27, 1057-1066 (1984) which are all incorporated by reference herein in their entirety. specific illustrative compounds within this class include Raloxifene [6-hydroxy-2-(4-hydroxyphenyl)benzo[b]thien-3-yl][4-[2-(1-piperidinyl)ethoxy]phenyl]methanone hydrochloride, formerly keoxifene; and [6-hydroxy-2-(4-hydroxyphenyl)benzo[b]thien-3-yl][4-[2-(1-pyrrolidinyl)ethoxy]phenyl]methanone Hydrochloride.

The 2-phenyl-3-aroylbenzo[b]thiophenes are exemplified by those in U.S. Pat. No. 4,133,814 and have the formula

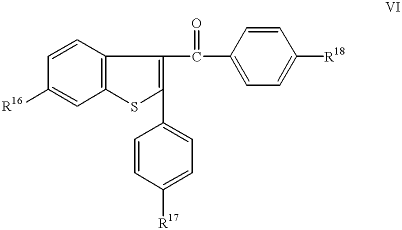

where

R¹⁶ is hydrogen, hydroxy, C₁-C₅ alkoxy, C₁-C₇ alkanoyloxy, C₃-C₇ cycloalkanoyloxy, (C₁-C₆ alkoxy)-C₁-C₇ alkanoyloxy, substituted or unsubstituted aroyloxy, or substituted or unsubstituted aryloxycarbonyloxy;

R¹⁷ is hydrogen, hydroxy, C₁-C₅ alkoxy, adamantoyloxy, chloro, bromo, C₁-C₇ alkanoyloxy, C₃-C₇ cycloalkanoyloxy, (C₁-C₆ alkoxy)-C₁-C₇ alkanoyloxy, substituted or unsubstituted aroyloxy, or substituted or unsubstituted aryloxycarbonyloxy;

R¹⁸ is —O—CH₂—CH₂—X′—NR¹⁹R²⁰; X′ is a bond or —CH₂—, R¹⁹ and R²⁰ are independently C₁-C₄ alkyl or are taken together with the nitrogen atom to which they are bonded to constitute a pyrrolidinyl, piperidinyl, hexamethyleneiminyl, or morpholinyl ring; and pharmaceutically acceptable acid addition salts and solvates thereof.

Methods of synthesizing these compounds are disclosed in U.S. Pat. No. 4,133,814. Raloxifene, and its preparation, are described in U.S. Pat. No. 4,418,068.

A fifth class of compounds comprises the 1-substituted-2-aryl-dihydronaphthalenes. These compounds are disclosed in and prepared according to procedures described in U.S. Pat. Nos. 4,400,543; 4,323,707; 4,230,862; and 3,274,213 which are all incorporated by reference herein in their entirety. Specific illustrative compounds within this class include Nafoxidene and Trioxifene.

The 1-substituted-2-aryl-dihydronaphthalenes are exemplified by U.S. Pat. No. 4,230,862 that describes compounds having the formula:

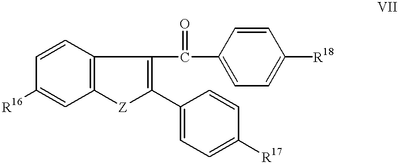

where

Z is —CH₂—CH₂— or —CH═CH—;

R¹⁶ is hydrogen, hydroxy or C₁-C₅ alkoxy;

R¹⁷ is hydrogen, hydroxy, C₁-C₅ alkoxy, C₁-C₅ acyloxy, C₁-C₅ alkoxycarbonyloxy, benzyloxy, adamantoyloxy, chloro, or bromo

R¹⁸ is C₁-C₅ alkoxy or —O—CH₂—CH₂—NR¹⁹R²⁰; and R¹⁹ and R²⁰ are independently C₁-C₄ alkyl or are taken together with the nitrogen atom to which they are bonded to constitute a pyrrolidinyl, piperidinyl, hexamethyleneimino, or morpholinyl ring; and pharmaceutically acceptable acid addition salts and solvates thereof.

Methods of synthesizing these compound are disclosed in U.S. Pat. No. 4,230,862.

The 1-substituted-2-aryl-dihydronaphthalenes are also exemplified by U.S. Pat. No. 3,274,213 that describes compounds having the formula

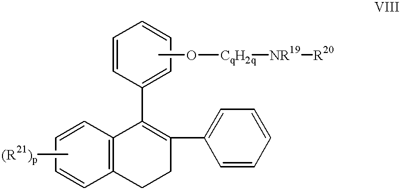

where

R¹⁹ and R²⁰ are C₁-C₈ alkyl or are taken together with the nitrogen atom to which they are bonded to form a 5 to 7 membered saturated heterocyclic radical selected from pyrrolidinyl, 2-methylpyrrolidinyl, 2,2 dimethylpyrrolidinyl, piperazinyl, 4-methylpiperazinyl, 2,4-dimethylpiperazinyl, morpholinyl, piperidinyl, 2-methylpiperidinyl, 3-methylpiperidinyl, hexamethyleneiminyl, homopiperazinyl, and homomorpholinyl;

q is 2 to 6;

p is 1 to 4;

R²¹ is C₁-C₈ alkoxy; and

pharmaceutically acceptable salts and solvates thereof.

Methods of synthesizing these compounds are disclosed therein.

A sixth class of compounds comprises the 2-substituted-3-aryl-benzofurans. These compounds are disclosed in and prepared according to procedures described in Teo et al., J. Med. Chem., 35, 1330-1339 which is incorporated by reference herein in its entirety.

The 2-substituted-3-aryl-benzofurans described in Teo et al., J. Med. Chem., 35, 1330-1339 (1992) includes those having the formula

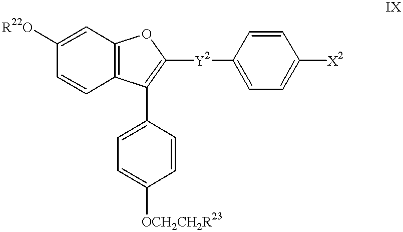

where

X² is halo;

Y² is a bond or —CH₂—;

R²² is hydrogen or methyl;

R²³ is a group —NR¹⁹R²⁰, where R¹⁹ and R²⁰ are independently C₁-C₄ alkyl or are taken together with the nitrogen atom to which they are bonded to constitute a pyrrolidinyl, piperidinyl, hexamethyleneiminyl or morpholinyl ring; and

pharmaceutically acceptable salts and solvates thereof.

Methods of synthesizing these compound are also disclosed therein.

The preferred class of compounds useful in the methods of the present invention are the benzothiophenes. More preferred are benzothiopenes having the formula:

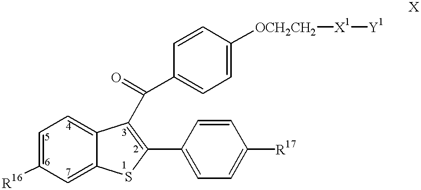

wherein

X¹ is a bond or —CH₂—;

R¹⁶ is hydroxyl, methoxy, C₁-C₇ alkanoyloxy, C₃-C₇ cycloalkanoyloxy, (C₁-C₆ alkoxy)-C₁-C₇ alkanoyloxy, substituted or unsubstituted aroyloxy, or substituted or unsubstituted aryloxycarbonyloxy;

R¹⁷ is hydrogen, hydroxyl, chloro, bromo, methoxy, C₁-C₇ alkanoyloxy, C₃-C₇ cycloalkanoyloxy, (C₁-C₆ alkoxy)-C₁-C₇ alkanoyloxy, substituted or unsubstituted or aroyloxy, or substituted or unsubstituted aryloxycarbonyloxy;

Y¹ is a heterocyclic ring selected from the group consisting of pyrrolidinyl, piperidinyl, or hexamethyleneiminyl; and pharmaceutically acceptable salts and solvates thereof. Particularly preferred are raloxifene and its pyrrolidinyl analog.

Included within the scope of this invention are salt formation products, preferably consisting of one molecule of the acidic bis-phosphonate and one molecule of the basic compound of the first group. Preferred salts are raloxifene/alendronate; raloxifene/pamidronate; raloxifene/risedronate; raloxifene/cycloheptyl amino methylidene bisphosphonate, and raloxifene/3-pyrolidenyl-1-hydroxy propylidene bisphosphonate.

The compounds utilized in the method of the present invention are effective over a wide dosage range. For example, dosages of compounds of the first group per day will normally fall within the range of about 0.01 to about 1000 mg/kg of body weight. In the treatment of adult humans, the range of about 10 to about 600 mg/day, in single or divided doses, is preferred. The amount of bisphonate will fall within 5 mg/day at 400 mg/day. However, it will be understood that the amount of the compounds actually administered will be determined by a physician in light of the relevant circumstances including the condition to be treated, the choice of compounds to be administered, the age, weight, and response of the individual patient, the severity of the patient's symptoms and the chosen route of administration. Therefore, the above dosage ranges are not intended to limit the scope of the invention in any way. While the present compounds are preferably administered orally, the compounds may also be administered by a variety of other routes such as the transdermal, subcutaneous, intranasal, intramuscular and intravenous routes.

While it is possible to administer the compounds directly, the compounds are preferably employed in the form of a pharmaceutical formulation comprising a pharmaceutically acceptable carrier, diluent or excipient and a compound of the invention. Such formulations will contain from about 0.01 percent to about 99 percent of the compounds.

In making the formulations of the present invention, the active ingredients will usually be mixed with at least one carrier, or diluted by at least one carrier, or enclosed within a carrier which may be in the form of a capsule, sachet, paper or other container. When the carrier serves as a diluent, it may be a solid, semi-solid or liquid material which acts as a vehicle, excipient or medium for the active ingredient. Thus, the formulations can be in the form of tablets, granules, pills, powders, lozenges, sachets, cachets, elixirs, emulsions, solutions, syrups, suspensions, aerosols (as a solid or in a liquid medium) and soft and hard gelatin capsules.

Examples of suitable carriers, diluents and excipients include lactose, dextrose, sucrose, sorbitol, mannitol, starches, gum acacia, calcium phosphate, alginates, liquid paraffin, calcium silicate, microcrystalline cellulose, polyvinyl pyrrolidone, cellulose, tragacanth, gelatin, syrup, methyl cellulose, methyl- and propyl-hydroxybenzoates, vegetable oils, such as olive oil, injectable organic esters such as ethyl oleate, talc, magnesium stearate, water and mineral oil. The formulations may also include wetting agents, lubricating, emulsifying and suspending agents, preserving agents, sweetening agents, perfuming agents, stabilizing agents or flavoring agents. The formulations of the invention may be formulated so as to provide quick, sustained or delayed release of the active ingredient after administration to the patient by employing procedures well-known in the art.

For oral administration, the compounds can be admixed with carriers and diluents and molded into tablets or enclosed in gelatin capsules.

The compositions are preferably formulated in a unit dosage form, each dosage containing from about 1 to about 500 mg, more usually about 5 to about 300 mg, of the active ingredient. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier, diluent or excipient therefor.

More particularly, there are three different, basic formulations envisioned within this invention.

1) Separate, Co-administered Dosage Formulations

This formulation consists of each drug separately formulated for parenteral or oral adminstration in manners well known to those skilled in the art and as particularly taught in references of each of the compounds cited. Because two separate formulations are being co-adminstered, each formulation, especially those by the oral route, would be color-coded or otherwise easily indentifiably labelled to avoid confusion by both patient or physician. Since a concept of this invention is to minimize the exposure of the patient to high doses of the bisphosphonate while maximizing the efficacy, the envisioned protocols for use of this invention would necessiate a short term or cyclic use of the bis-phosphonate and a continous use of a compound of the first group.

2) Single Mixture Formulations

One way to avoid possible confusion and which would allow for various strengths of the two different drugs would be to combine the two entities in a simple mixture in forms well known and taught in the art. A patient could an orange tablet containing 50 mg of, for example, raloxifene and 25 mg of risedronate, once a day, for two weeks, followed in continuance of the blue tablet of 50 mg of raloxifene.

3) Single Molar Defined Salt Formulations

This formulation, where each drug is preferably the counter ion for the other, would lead to a salt of defined chemical composition. This would aid in consistancy and homogeneity of the preparation and may aid in absorption of the bis-phosphonate by oral adminstration.

Since there is a great deal of concern about the side-effects of bis-phosphonate therapy, prolonged, continous use of the bis-phosphonate would not be recommended, rather a cyclic regiment would be more appropriate. An example of such a cyclic protocol is taught in the art in regard to the use of bis-phosphonates in the treatment of Paget's Disease and specifically for the treatment of osteoporosis in “Watts, N. B., et al., The New England J. of Medicine, 323(2), p.73-79.

In order to more fully illustrate the operation of this invention, the following examples of formulations are provided. The examples are illustrative only and are not intended to limit the scope of the invention. The formulations may employ as active ingredients any of the compounds described above.

### FORMULATION 1

Hard gelatin capsules are prepared using the following ingredients:

| | Amt. per | Concentration by |
|---|---|---|
| | Capsule | Weight (percent) |
| Active Ingredient(s) | 250 mg | 55.0 |
| Starch dried | 220 mg | 43.0 |
| Magnesium stearate | 10 mg | 2.0 |
| | 460 mg | 100.0 |

The above ingredients are mixed and filled into hard gelatin capsules in 460 mg quantities.

### FORMULATION 2

Capsules each containing 20 mg of medicament are made as follows:

| | Amt. per | Concentration by |
|---|---|---|
| | Capsule | Weight (percent) |
| Active Ingredient(s) | 20 mg | 10.0 |
| Starch | 89 mg | 44.5 |
| Microcrystalline | 89 mg | 44.5 |
| cellulose | | |
| Magnesium stearate | 2 mg | 1.0 |
| | 200 mg | 100.0 |

The active ingredient(s), cellulose, starch and magnesium stearate are blended, passed through a No. 45 mesh U.S. sieve and filled into a hard gelatin capsule.

### FORMULATION 3

Capsules each containing 100 mg of active ingredient(s) are made as follows:

| | Amt. per | Concentration by |
|---|---|---|
| | Capsule | Weight (percent) |
| Active Ingredient(s) | 100 mg | 29.0 |
| Polyoxyethylenesorbitan | 50 mcg | 0.02 |
| monooleate | | |
| Starch powder | 250 mg | 71.0 |
| | 250.05 mg | 100.02 |

The above ingredients are thoroughly mixed and placed in an empty gelatin capsule.

### FORMULATION 4

Tablets each containing 10 mg of active ingredient(s) are made up as follows:

| | Amt. per | Concentration by |
|---|---|---|
| | Capsule | Weight (percent) |
| Active Ingredient(s) | 10 mg | 10.0 |
| Starch | 45 mg | 45.0 |
| Microcrystalline | 35 mg | 35.0 |
| cellulose | | |
| Polyvinyl | 4 mg | 4.0 |
| pyrrolidone (as 10% | | |
| solution in water) | | |
| Sodium carboxyethyl | 4.5 mg | 4.5 |
| starch | | |
| Magnesium stearate | 0.5 mg | 0.5 |
| Talc | 1 mg | 1.0 |
| | 100 mg | 100.0 |

The active ingredient(s), starch and cellulose are passed through a No. 45 mesh U.S. sieve and mixed thoroughly. The solution of polyvinylpyrrolidone is mixed with the resultant powders which are then passed through a No. 14 mesh U.S. sieve. The granule so produced is dried at 50°-60° C. and passed through a No. 18 mesh U.S. sieve. The sodium carboxymethyl starch, magnesium stearate and talc, previously passed through a No. 60 mesh U.S. sieve, are then added to the granule which, after mixing, is compressed on a tablet machine to yield a tablet weighing 100 mg.

### FORMULATION 5

A tablet formula may be prepared using the ingredients below:

| | Amt. per | Concentration by |
|---|---|---|
| | Capsule | Weight (percent) |
| Active Ingredient(s) | 250 mg | 38.0 |
| Cellulose | 400 mg | 60.0 |
| microcrystalline | | |
| Silicon dioxide | 10 mg | 1.5 |
| fumed | | |
| Stearic acid | 5 mg | 0.5 |
| | 665 mg | 100.0 |

The components are blended and compressed to form tablets each weighing 665 mg.

### FORMULATION 6

Suspensions each containing 5 mg of medicaments per 40 ml dose are made as follows:

| | Per 5 ml of suspension | |
|---|---|---|
| Active Ingredient(s) | 5 | mg |
| Sodium carboxymethyl | 50 | mg |
| cellulose | | |
| Syrup | 1.25 | ml |
| Benzoic acid solution | 0.10 | ml |
| Flavor | q.v. | |
| Color | q.v. | |

| | |
|---|---|
| Water | q.s. to 5 ml |

The medicament is passed through a No. 45 mesh U.S. sieve and mixed with the sodium carboxymethylcellulose and syrup to form a smooth paste. The benzoic acid solution, flavor and color is diluted with some of the water and added, with stirring. Sufficient water is then added to produce the required volume.

### FORMULATION 7

An aerosol solution is prepared containing the following components:

| | Concentration by Weight (%) |
|---|---|
| Active Ingredient(s) | 0.25 |
| Ethanol | 29.75 |
| Propellant 22 | 70.00 |
| (Chlorodifluoromethane) | |
| | 100.00 |

The active compound(s) are mixed with ethanol and the mixture added to a portion of the propellant 22, cooled to −30° C. and transferred to a filling device. The required amount is then fed to a stainless steel container and diluted further with the remaining amount of propellant. The valve units are then fitted to the container.

The following is an example of the preparation of a salt formula between a bisphosphonate and a compound from group 1.

### Combination Salt of [2-(4-Hydroxyphenyl)-6-hydroxybenzo[B]thien-3-yl][4-[2-(1-piperidenyl)ethoxy]phenyl]methanone and 4-amino-1-hydroxybutyl-1,1-bis phosphonate

2.37 g of [2-(4-hydroxyphenyl)-6-hydroxybenzo[B]thien-3-yl]4-[2-(1-piperidenyl)ethoxy]phenyl]methanone (0.005 mol) was dissolved in 25 mL of EtOH.

1.36 g of 4-amino-1-hydroxybutyl-1,1-bisphosphonate mono sodium (0.005 mol) was dissolved in 25 mL of water and 5 mL of 1N HCl (0.005) was added. The reaction mixture was evaporated to a gummy white solid and redissolved in 10 mL of water. This aqueous solution was then added to the EtOH solution prepared above. This reaction mixture was heated on a steam bath for one hour in order to dissolve all the components. The reaction mixture was evaporated to a white amorphous powder and dried under vaccuum at room temperature for 24-hours.

This yielded 3.6 g of the title compound as a white amorphous powder.

MS: m/e=780 (M-1).

474 (Raloxifene base +1)

309 (Bis-phosphonate-NaCl +1)

EA: Clac: C, 49.21; h,5.16; N,3.59 Found: C,41.80; H, 5.06; N,3.57.

In the following, a model of post-menopausal osteoporosis was used in which effects of different treatments upon femur density were determined.

Seventy-five day old female Sprague Dawley rats (weight range of 225 to 275 g) were obtained from Charles River Laboratories (Portage, Mich.). They were housed in groups of 3 and had ad libitum access to food (calcium content approximately 1%) and water. Room temperature was maintained at 22.2°±1.7° C. with a minimum relative humidity of 40%. The photoperiod in the room was 12 hours light and 12 hours dark.

One week after arrival, the rats underwent bilateral ovariectomy under anesthesia (44 mg/kg Ketamine and 5 mg/kg Xylazine (Butler, Indianapolis, Ind.) administered intramuscularly). Treatment with vehicle or the indicated compound was initiated either on the day of surgery following recovery from anesthesia or 35 days following the surgery.

Oral dosage was by gavage in 0.5 mL of 1% carboxymethylcellulose (CMC).

Body weight was determined at the time of surgery and weekly during the study, and the dosage was adjusted with changes in body weight. Vehicle-treated ovariectomized (ovex) rats and non-ovariectomized (intact) rats were evaluated in parallel with each experimental group to serve as negative and positive controls.

The rats were treated daily for 35 days (6 rats per treatment group) and sacrificed by decapitation on the 36th day. The 35-day time period was sufficient to allow maximal reduction in bone density, measured as described infra. At the time of sacrifice, the uteri were removed, dissected free of extraneous tissue, and the fluid contents were expelled before determination of wet weight in order to confirm estrogen deficiency associated with complete ovariectomy. Uterine weight was routinely reduced about 75% in response to ovariectomy. The uteri were then placed in 10% neutral buffered formalin to allow for subsequent histological analysis.

The right femurs are excised and scanned at the distal metaphysis proximal from the growth plate region by grey scale image analysis of digitalized X-ray generation on a Nicolet NXR-1200 real time X-ray imaging system. Additional image analysis was performed with NIH Image (1.45) software Relative density of bone was assayed over the lower end of the grey scale (therefore highest density range to demonstrate activity).

One of the major concepts put forth in the IDM of the use of Raloxifene and Bis-phosphonates for the treatment of osteoporosis was the concept of limiting the exposure of the patient to the potential side-effects of bis-phosphonates. We have done some further studies combinding other hormonally acting drugs with bis-phosphonates to see if Raloxifene has a unique profile when used in this modality. The data presented below demonstrate that raloxifene when used in combination with alendronate does have a different and more beneficial profile.

In Table 1, Alendronate (ALN) was combind with Provera (a synthetic Progestin) and tested in the 5-week ovex rat model of post-menopausal osteoporosis. As can be seen, at the two doses of Provera (1 and 10 mg/kg) and ALN at 0.1 mg/kg, there was no protection from bone loss. The high dose of ALN (1 mg/kg) had to be used to gain protection, even though at 10 mg/kg of provera had a protective effect by itself. This in contrast to RAL and ALN (93-11), where the low dose of RAL (0.1 mg/kg) and ALN (0.1 mg/kg) gave some protection from bone loss. At the high doses of either compound when given in combination, there seems to be little contribution by the Provera.

In Table 2, Alendronate was combined with ethynylestradiol (EE₂, a synthetic estrogen), these results were similar to the raloxifene and alendronate combination, except that the total protection with Raloxifene and alendronate was superior. Again keeping in mind the concept of limiting the exposure to alendronate, EE₂ at both 30 and 100 ug/kg and alendronate at 0.1 mg/kg could not achieve the complete protection of bone loss seen with the intact controls. The dose of Alendronate had to be increased to see that level of activity. In contast, in Table 3, virtually complete protection could be achieved with Raloxifene at 1 mg/kg and alendronate at 0.1 mg/kg.

In summary, each of the four agents tested could afford some level of protection against bone loss in this model when used alone. Alendronate and Provera demonstrated the least interactive effects. EE₂ and raloxifene plus alendronate did show interactive effects. However, raloxifene and alendronate in combination demonstrated the greatest protection from bone loss with the lowest exposure to the potentially undesirable side-effects of alendronate.

(It should be noted that when viewing the attached biological data @0.1 mg/kg of alendronate and 0.1 mg/kg of raloxifene, this combination gave good anti-resorptive activity even though each compound separately was inactive. The molar ratio of the two compounds was 1:2 (raloxifene:alendronate) in this assay. It would seem very likely that a salt form with a 1:1 molar ratio, given at a slightly higher dose would be effective, whereas the two compounds if given separately might not.)

TABLE 1

| X-RAY-VOLTAGE/CONTRAST | | 47KV/4.5C | |
|---|---|---|---|
| | | RANGE | ± STD. |
| GROUP | DOSE | 0-50 | ERR |
| INTACT | | 58.81* | ±7.55 |
| OVEX | CDX PO | 19.85 | ±6.27 |
| CONTROL | | | |
| ALN | 0.1 mg/kg PO CDX | 37.47 | ±7.08 |
| ALN | 1 mg/kg PO CDX | 73.79* | ±7.73 |
| Provera | 1 mg/kg PO CDX | 30.10 | ±5.61 |
| Provera | 10 mgmg/kg PO CDX | 64.16* | ±6.78 |
| ALN + Provera | 0.1 mg/kg + 1 mg/kg PO CDX | 35.46 | ±4.85 |
| ALN + Provera | 0.1 mg/kg + 10 mg/kg PO CDX | 38.89 | ±9.67 |
| ALN + Provera | 1 mg/kg + 10 mg/kg PO CDX | 100.81* | ±4.43 |
| ALN + Provera | 1 mg/kg + 10 mg/kg PO CDX | 105.87* | ±2.92 |
| RAL | 1 mg/kg PO CDX | 57.67* | ±10.25 |
| EE2 | 100 μg/kg PO CDX | 51.29* | ±6.07 |

*P <= .05, TWO TAILED STUDENT T ON RAW DATA

TABLE 2

| X-RAY-VOLTAGE CONTRAST | | 47KV/4.5C | |
|---|---|---|---|
| | | RANGE | ± STD. |
| GROUP | DOSE | 0-50 | ERR |
| INTACT | | 84.40* | ±7.70 |
| OVEX | CDX PO | 34.24 | ±7.53 |
| CONTROL | | | |
| ALN | 0.1 mg/kg PO CDX | 39.47 | ±2.39 |
| ALN | 1 mg/kg PO CDX | 65.60* | ±3.71 |
| EE2 | 100 μg/kg PO CDX | 61.09* | ±6.72 |
| EE2 | 30 μg/kg PO CDX | 50.87 | ±7.89 |
| ALN + EE2 | 0.1 mg/kg + 30 μg/kg PO CDX | 57.82* | ±5.86 |
| ALN + EE2** | 0.1 mg/kg + 100 μg/kg PO | 51.14 | ±11.09 |
| | CDX | | |
| ALN + EE2 | 1 mg/kg + 30 μg/kg PO CDX | 86.93* | ±4.71 |
| ALN + EE2 | 1 mg/kg + 100 μg/kg PO CDX | 97.54* | ±3.37 |
| RAL | 1 mg/kg PO CDX | 80.98* | ±5.25 |

*P <= .05, TWO TAILED STUDENT T ON RAW DATA
**1/5 DEAD

TABLE 3

| X-RAY-VOLTAGE CONTRAST | | 47KV/4.5C | |
|---|---|---|---|
| | | RANGE | ± STD. |
| GROUP | DOSE | 0-60 | ERR |
| INTACT | | 71.72* | ±5.66 |
| OVEX | CDX PO | 31.49 | ±7.27 |
| CONTROL | | | |
| ALN | 0.1 mg/kg PO CDX | 47.25 | ±6.15 |
| ALN | 1 mg/kg PO CDX | 78.61* | ±7.26 |
| RAL | 0.1 mg/kg PO CDX | 36.76 | ±6.36 |
| RAL | 1 mg/kg PO CDX | 40.37 | ±3.27 |
| RAL + ALN | 0.1 mg/kg + 0.1 mgkg PO CDX | 59.41* | ±9.12 |
| RAL + ALN | 0.1 mg/kg + 1 mg/kg PO CDX | 104.58* | ±7.97 |
| RAL + ALN | 1 mg/kg + 0.1 mg/kg PO CDX | 67.21* | ±6.77 |
| RAL + ALN | 1 mg/kg + 1 mg/kg PO CDX | 95.62* | ±4.02 |
| EE2 | 100 μg/kg PO CDX | 62.88* | ±11.34 |

*P <= .05, TWO TAILED STUDENT T ON RAW DATA

TABLE 4

| BBA-9311 | | Ave BW | SE BW | | Ave Ut | SE Ut | Ut t- |
|---|---|---|---|---|---|---|---|
| Grp | Cmpd & Mg/kg | Chg | Chg | BW t-Test | Wt | Wt | Test |
| 1 | ALN - .1 mg/kg | 102.167 | 8.3842 | 0.3147 | 100.833 | 4.8677 | 0.3558 |
| 2 | ALN - 1 mg/kg | 106.000 | 7.4744 | 0.4176 | 108.667 | 8.8531 | 0.3116 |
| 3 | RAL - .1 mg/kg | 75.833 | 8.1626 | 0.0146 | 157.667 | 15.1166 | 0.0034 |
| 4 | RAL - 1 mg/kg | 49.667 | 9.1930 | 0.0007 | 166.000 | 12.5804 | 0.0005 |
| 5 | RAL - .1 mg/kg + ALN - .1 mg/kg | 59.000 | 8.3427 | 0.0017 | 179.000 | 9.9432 | 0.0001 |
| 6 | RAL - .1 mg/kg + ALN - 1 mg/kg | 59.000 | 9.5638 | 0.0025 | 157.500 | 9.0508 | 0.0002 |
| 7 | RAL - 1 mg/kg + ALN - .1 mg/kg | 56.500 | 4.2170 | 0.0003 | 192.333 | 17.2040 | 0.0003 |
| 8 | RAL - 1 mg/kg + ALN - 1 mg/kg | 49.667 | 4.2635 | 0.0002 | 203.500 | 8.1312 | 0.0001 |
| 9 | EE2 100 μg/kg | 49.000 | 2.0000 | 0.0001 | 399.000 | 38.4309 | 0.0001 |
| 10 | ovex | 108.667 | 10.0089 | | 103.500 | 5.0448 | |
| 11 | intact | 62.833 | 4.3697 | 0.0009 | 562.833 | 59.2646 | 0.0001 |

Procedure:
Animals ovariectomized and shipped by supplier the day before initiation of treatment. Body weight determined
and solutions prepared weekly and adjusted to body weight change. Right femur, tibia, uteus and blood collected
at sacrifice.
Schedule:
Start/treat.: group #1-6 on 6/1/93, #7-11 on 6/4/93
Sacrifice: group #1-6 on 7/5/93, #7-11 on 7/8/93
Column E, H and K = t-test values, unpaired, one tail, ovex vs group using StatView, P =< .05
ANIMALS FASTED 12 HRS. PRIOR TO SACRIFICE

We claim:

1. A method of inhibiting bone loss comprising administering to a human in need thereof a first compound selected from 1) triarylethylenes; 2) 2,3-diaryl-2H-1-benzopyrans; 3) 1-aminoalkyl-2-phenylindoles; 4) 2-phenyl-3-aroylbenzothiophenes; 5) 1-substituted-2-aryl-dihydronaphthalenes; or 6) benzofurans; and a second compound being a bisphosphonate; or pharmaceutically acceptable salts and solvates thereof.

2. The method of claim 1 wherein said bisphosphnate is selected from alendronate, pomidronate, risedronate, cycloheptyl amino methyl idine bisphosphonate, and 3-pyrolidonyl-1-hydroxy propylidene bisphosphonate.

3. The method of claim 1 wherein said first compound is a triarylethylene and pharmaceutically acceptable salts and solvates thereof.

4. The method of claim 3 wherein said triarylethylene has the formula 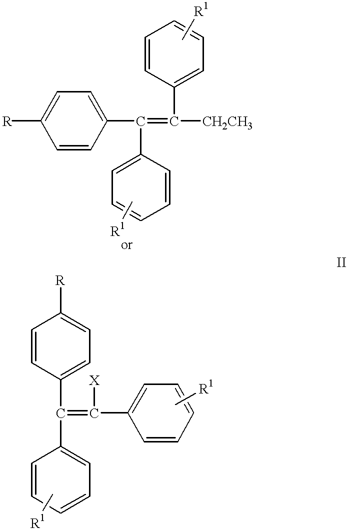
where
R is a basic ether group of the formula —OCₙH₂ₙA; n is 2,3 or 4 and A is a dialkylamino group where the alkyl group contains from 1 to 4 carbon atoms or a cyclic structure selected from N-piperidinyl, N-pyrrolidinyl, N-hexamethyleneimino and N-morpholinyl group; and each R¹ is independently hydrogen, hydroxy, halogen or methoxy; and X is halogen;
or 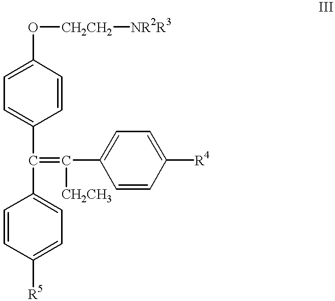
where
R² and R³ are independently selected from hydrogen and methyl;
R⁴ is isopropyl, isopropen-2-yl, or mono or dihydroxy isopropyl;
R⁵ is hydroxy or phosphate (—OPO₃H₂);
and pharmaceutically acceptable salts and solvates thereof.

5. The method of claim 4 wherein said triarylethylene has the formula: 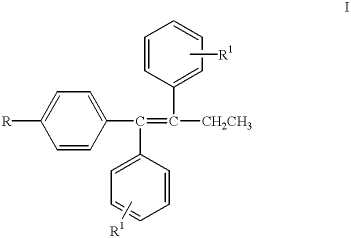
where R is a basic ether group of the formula —OCₙH₂ₙA; n is 2, 3 or 4 and A is a dialkylamino group where the alkyl groups independently contain from 1 to 4 carbon atoms or a cyclic structure selected from N-piperidinyl, N-pyrrolidinyl, N-morpholinyl, and N-hexamethyleneimino; each R¹ is independently hydrogen, hydroxy, halogen or methoxy; and pharmaceutically acceptable salts and solvates thereof.

6. The method of claim 5 wherein said triarylethylene has the formula 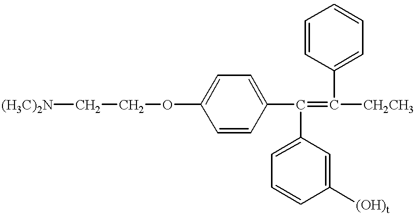
wherein t is 1 or 0; and pharmaceutically acceptable salts and solvates thereof.

7. The method of claim 4 wherein said triarylethylene has the formula 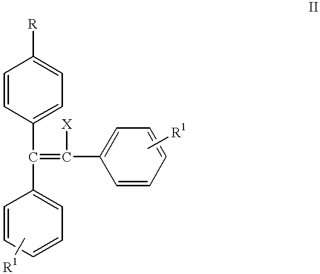
where
R is a basic ether group of the formula —OCₙH₂ₙA; n is 2,3 or 4 and A is a dialkylamino group where the alkyl group contains from 1 to 4 carbon atoms or a cyclic structure selected from N-piperidinyl, N-pyrrolidinyl and N-morpholinyl group; and each R¹ is independently hydrogen, halogen or methoxy; and X is halogen; and pharmaceutically acceptable salts and solvates thereof.

8. The method of claim 7 wherein said triarylethene has the formula 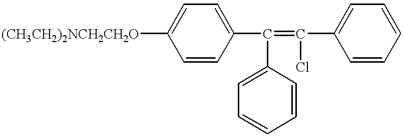
and pharmaceutically acceptable salts and solvates thereof.

9. The method of claim 1 wherein said first compound is a 2,3-diaryl-2H-1-benzopyran and pharmaceutically acceptable salts and solvates thereof.

10. The method of claim 9 wherein said 2,3-diaryl-2H-1-benzopyran has the formula 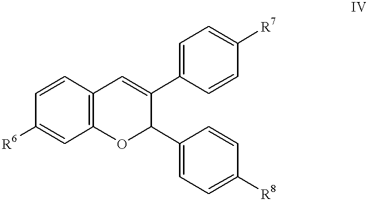
where
R⁶ and R⁷ are the same or different hydrogen hydroxy, C₁-C₁₇ alkoxy or C₂-C₁₈ alkanoyloxy;
R8 is 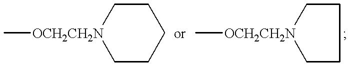
and pharmaceutically acceptable salts and solvates thereof.

11. The method of claim 10 wherein said 2,3-diaryl-2H-1-benzopyran is selected from 2-[4-[2-(1-piperidinyl)ethoxy]phenyl]-3-[4-hydroxyphenyl]-2H-1-benzopyran; 2-[4-[2-(1-piperidinyl)ethoxy]phenyl]-3-phenyl-7-methoxy-2H-1-benzopyran; 2-[4-[2-(1-piperidinyl)ethoxy]phenyl]-3-[4-hydroxyphenyl]-7-hydroxy-2H-1-benzopyran, and pharmaceutically acceptable salts and solvates thereof.

12. The method of claim 1 wherein said first compound is a 1-aminoalkyl-2-phenylindole and pharmaceutically acceptable salts and solvates thereof.

13. The method of claim 12 wherein said 1-aminoalkyl-2-phenylindole has the formula 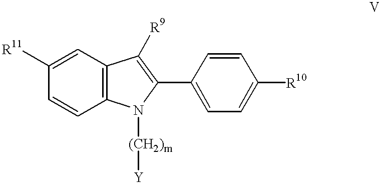
where
R⁹ is hydrogen or methyl;
R¹⁰ and R¹¹ are methoxy or hydroxy;
m is 4 to 8;
Y is NR¹²R¹³ where R¹² and R¹³ are independently selected from hydrogen, methyl and ethyl or one of R¹² or R¹³ is hydrogen and the other is benzyl or are combined with the nitrogen atom to constitute a pyrrolidinyl, piperidinyl or morpholinyl group;
and pharmaceutically acceptable salts and solvates thereof.

14. The method of claim 1 wherein said first compound is a 2-phenyl-3-aroylbenzo[b]thiophene and pharmaceutically acceptable salts and solvates thereof.

15. The method of claim 14 wherein said 2-phenyl-3-aroylbenzo[b]thiophene has the formula 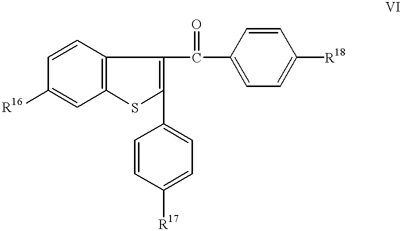
where
R¹⁶ is hydrogen, hydroxy or C₁-C₅ alkoxy C₁-C₇ alkanoyloxy, C₃-C₇ cycloalkanoyloxy, (C₁-C₆ alkoxy)-C₁-C₇ alkanoyloxy, substituted or unsubstituted aroyloxy, or substituted or unsubstituted aryloxycarbonyloxy;
R¹⁷ is hydrogen, hydroxy, C₁-C₅ alkoxy, adamantoyloxy, chloro, bromo, C₁-C₇ alkanoyloxy, C₃-C₇ cycloalkanoyloxy, (C₁-C₆ alkoxy)-C₁-C₇ alkanoyloxy, substituted or unsubstituted aroyloxy, or substituted or unsubstituted aryloxycarbonyloxy;
R¹⁸ is —O—CH₂—CH₂—X′—NR¹⁹R²⁰;
X′ is a bond or —CH₂—, R¹⁹ and R²⁰ are independently C₁-C₄ alkyl or are taken together with the nitrogen atom to which they are bonded to constitute a pyrrolidinyl, piperidinyl, hexamethyleneiminyl, or morpholinyl ring; and pharmaceutically acceptable acid addition salts and solvates thereof.

16. The method of claim 15 wherein said 2-phenyl-3-aroylbenzo[b]thiophene has the formula 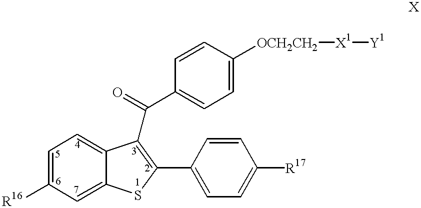
wherein
X¹ is a bond or —CH₂—;
R¹⁶ is hydroxyl, methoxy, C₁-C₇ alkanoyloxy, C₃-C₇ cycloalkanoyloxy, (C₁-C₆ alkoxy)-C₁-C₇ alkanoyloxy, substituted or unsubstituted aroyloxy, or substituted or unsubstituted aryloxycarbonyloxy;
R¹⁷ is hydrogen, hydroxyl, chloro, bromo, methoxy, C₁-C₇ alkanoyloxy, C₃-C₇ cycloalkanoyloxy, (C₁-C₆ alkoxy)-C₁-C₇ alkanoyloxy, substituted or unsubstituted or aroyloxy, or substituted or unsubstituted aryloxycarbonyloxy;
Y¹ is a heterocyclic ring selected from the group consisting of pyrrolidinyl, piperidinyl, or hexamethyleneiminyl; and pharmaceutically acceptable salts and solvates thereof.

17. The method of claim 16 wherein said 2-phenyl-3-aroylbenzo[b]thiophene is [6-hydroxy-2-(4-hydroxyphenyl)benzo[b]thien-3-yl][4-[2-(1-piperidinyl)ethoxy]phenyl]methanone and, pharmaceutically acceptable salts and solvates thereof.

18. The method of claim 16 wherein said 2-phenyl-3-aroylbenzo[b]thiophene is and [6-hydroxy-2-(4-hydroxyphenyl)benzo[b]thien-3-yl][4-[2-(1-pyrrolidinyl)ethoxy]phenyl]methanone and pharmaceutically acceptable salts and solvates thereof.

19. The method of claim 1 wherein said first compound is a 1-substituted-2-aryl-dihydronaphthalene and pharmaceutically acceptable salts and solvates thereof.

20. The method of claim 19 wherein said 1-substituted-2-aryl-dihydronaphthalene has the formula 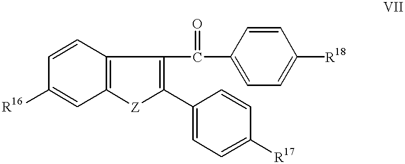
where
Z is —CH₂—CH₂— or —CH═CH—;
R¹⁶ is hydrogen, hydroxy or C₁-C₅ alkoxy;
R¹⁷ is hydrogen, hydroxy, C₁-C₅ alkoxy, C₁-C₅ acyloxy, C₁-C₅ alkoxycarbonyloxy, benzyloxy, adamantoyloxy, chloro, or bromo;
R¹⁸ is —O—CH₂—CH₂—NR¹⁹R²⁰; and R¹⁹ and R²⁰ are independently C₁-C₄ alkyl or are taken together with the nitrogen atom to which they are bonded to constitute a pyrrolidinyl, piperidinyl, hexamethyleneimino, or morpholinyl ring; subject to the limitation that when R¹⁷ is hydrogen, R¹⁶ is hydrogen, hydroxy, or C₁-C₅ alkoxy and at least one of R¹⁶ and R¹⁷ is other than hydrogen;
or 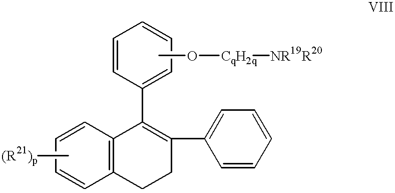
where
R¹⁹ and R²⁰ are C₁-C₈ alkyl or are taken together with the nitrogen atom to which they are bonded to form a 5 to 7 membered saturated heterocyclic radical selected from pyrrolidinyl, 2-methylpyrrolidinyl, 2,2 dimethylpyrrolidinyl, piperazinyl, 4-methylpiperazinyl, 2,4-dimethylpiperazinyl, morpholinyl, piperidinyl, 2-methylpiperidinyl, 3-methylpiperidinyl, hexamethyleneiminyl, homopiperazinyl, and homomorpholinyl;
q is 2 to 6;
p is 1 to 4;
R²¹ is C₁-C₈ alkoxy; and
pharmaceutically acceptable salts and solvates thereof.

21. The method of claim 20 wherein said 1-substituted-2-aryl-dihydronaphthalene has the formula 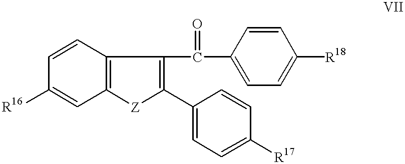
where
Z is —CH₂—CH₂— or —CH═CH—;
R¹⁶ is hydrogen, hydroxy or C₁-C₅ alkoxy;
R¹⁷ is hydrogen, hydroxy, C₁-C₅ alkoxy, C₁-C₅ acyloxy, C₁-C₅ alkoxycarbonyloxy, benzyloxy, adamantoyloxy, chloro, bromo
R¹⁸ is C₁-C₅ alkoxy or —O—CH₂—CH₂—NR¹⁹R²⁰; and R¹⁹ and R²⁰ are independently C₁-C₄ alkyl or are taken together with the nitrogen atom to which they are bonded to constitute a pyrrolidinyl, piperidinyl, hexamethyleneimino, or morpholinyl ring; and pharmaceutically acceptable salts and solvates thereof.

22. The method of claim 21 wherein said 1-substituted-2-aryl-dihydronaphthalene has the formula 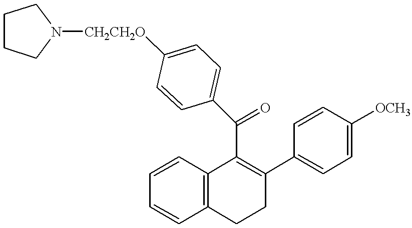
and pharmaceutically acceptable salts and solvates thereof.

23. The method of claim 20 wherein said 1-substituted-2-aryl-dihydronaphthalene has the formula 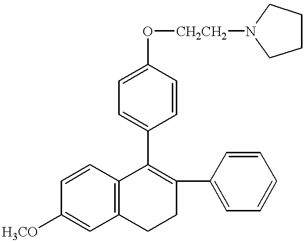
and pharmaceutically acceptable salts and solvates thereof.

24. The method of claim 1 wherein said first compound is a 2-substituted-3-aryl-benzofuran and pharmaceutically acceptable salts and solvates thereof.

25. The method of claim 24 wherein said 2-substituted-3-aryl-benzofuran has the formula 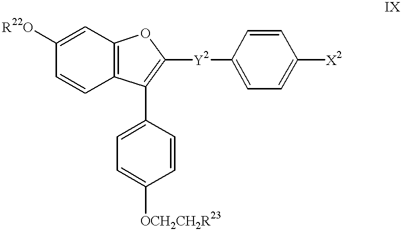
where
X² is halo;
Y² is a bond or —CH₂—;
R²² is hydrogen or methyl;
R²³ is a group —NR¹⁹R²⁰, where R¹⁹ and R²⁰ are independently C₁-C₄ alkyl or are taken together with the nitrogen atom to which they are bonded to constitute a pyrrolidinyl, piperidinyl, hexamethyleneiminyl or morpholinyl ring; and
pharmaceutically acceptable salts and solvates thereof.

26. A pharmaceutical formulation comprising a first compound selected from 1) triarylethylenes; 2) 2,3-diaryl-2H-1-benzopyrans, 3) 1-aminoalkyl-2-phenylindoles; 4) 2-phenyl-3-aroylbenzothiophenes, 5) 1-substituted-2-aryl-dihydronaphthalenes; or 6) benzofuran, and a second compound being a bisphosphonate: or pharmaceutically acceptable salts and solvates thereof, and one or more excipients, diluents and carriers therefor.

27. A combination salt comprising a first compound selected from 1) triarylethylenes; 2) 2,3-diaryl-2H-1-benzopyrans, 3) 1-aminoalkyl-2-phenylindoles; 4) 2-phenyl-3-aroylbenzothiophenes, 5) 1-substituted-2-aryl-dihydronaphthalenes; or 6) benzofuran, and a second compound being a bisphosphonate.

28. The salt of claim 27, wherein said first compound is raloxifene and said bisphosphonate is selected from alendronate, pamidronate, risedronate, cycloheptyl aminomethylidene bisphosphonate, or 3-pyrolidenyl-1-hydroxy propylidene bisphosphonate.

29. The salt of claim 28 wherein said first compound is raloxifene and said bisphosphonate is alendronate.
